(19) United States
(12) Patent Application Publication
(10) Pub. No.: US 2022/0062542 A1
(43) Pub. Date: Mar. 3, 2022

(54) SIMPLIFIED WEARABLE DRUG DELIVERY DEVICE

(71) Applicant: Insulet Corporation, Acton, MA (US)

(72) Inventors: David NAZZARO, Groveland, MA (US); Jason O'CONNOR, Acton, MA (US); Ian MCLAUGHLIN, Groton, MA (US); Philip HILLDALE, Raleigh, NC (US); Joon Bok LEE, Acton, MA (US); Bret CHRISTENSEN, Lexington, MA (US); Maureen MCCAFFREY, Arlington, MA (US); Thomas METZMAKER, Harvard, MA (US)

(21) Appl. No.: 17/412,666

(22) Filed: Aug. 26, 2021

Related U.S. Application Data
(60) Provisional application No. 63/072,417, filed on Aug. 31, 2020.
(60) Provisional application No. 63/071,196, filed on Aug. 27, 2020.

(51) Int. Cl. A61M 5/168; A61M 5/142; A61M 5/158; A61M 5/172

(52) U.S. Cl. CPC A61M 5/16877; A61M 5/14244; A61M 2205/502; A61M 5/172; A61M 2005/1585; A61M 5/158

(57) ABSTRACT

Embodiments of the present disclosure relate to techniques, processes, devices or systems for automating fluid delivery without the use of an external interface device. In one approach, a wearable drug delivery device may include a reservoir configured to store a liquid drug, a pump mechanism coupled to the reservoir and operable to expel the liquid drug from the reservoir, and a mechanical triggering device engageable by a user. The mechanical triggering device is operable to change between a first configuration and a second configuration to control deployment of a needle to deliver the liquid drug into a patient.

### CROSS REFERENCE TO RELATED APPLICATIONS

This application claims the benefit of U.S. Provisional Application Ser. No. 63/072,417, filed Aug. 31, 2020, and U.S. Provisional Application Ser. No. 63/071,196, Filed Aug. 27, 2020, the teachings of which are incorporated herein by reference.

### TECHNICAL FIELD

The disclosed embodiments generally relate to medication delivery. More particularly, the disclosed embodiments relate to techniques, processes, devices or systems for automating fluid delivery without the use of an external interface device.

### BACKGROUND

Patients with Type-1 diabetes often begin with multiple daily injections when first diagnosed with the disease, often with longer acting insulin. Some patients have difficulty transitioning to continuous subcutaneous insulin infusion (CSII) pump devices due to the significantly increased burden of care, such as need to generate clinical parameters, update the parameters in real time, prime/activate/deactivate the pump sites, among others. Therefore, some patients avoid using CSII pump devices with faster acting insulin despite the demonstrably improved standard of care.

Pre-programmed, basal-only delivery devices are one type of drug delivery device available to diabetic patients. These devices are typically factory configured, or include input devices, like buttons or touch screen displays, in conjunction with a GUI, to allow users to set therapy parameters. Devices which are factory configured are limited by a “one or two sizes fit all” approach, which prevents more specific user customization. Furthermore, devices which require patient or health care provider (HCP) input to set therapy leave opportunity for error and drive up overall cost of the device due to the extra system components.

Accordingly, there is a need for a simplified wearable drug delivery device that can provide insulin therapy to patients without the need for additional patient interactions using, e.g., an external interface device.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

In some approaches, a wearable drug delivery device may include a reservoir configured to store a liquid drug, a pump mechanism coupled to the reservoir and operable to expel the liquid drug from the reservoir, a mechanical triggering device engageable by a user, the mechanical triggering device operable to change between a first configuration and a second configuration to control deployment of a needle to deliver the liquid drug into a patient.

In some approaches, a method may include providing a drug delivery device including a reservoir operable to store liquid drug, coupling a pump mechanism to the reservoir, the pump mechanism operable to expel the liquid drug from the reservoir, and biasing a mechanical triggering device between a first configuration and a second configuration to control deployment of a needle to deliver the liquid drug into a patient.

In some approaches, a non-transitory computer readable medium embodied with programming code executable by a processor, and the processor when executing the programming code may be operable to perform functions to: receive an input from a mechanical triggering device communicably connected with a reservoir and a pump mechanism of a drug delivery device, wherein the input indicates deployment of a needle to deliver the insulin from the reservoir to a patient, and based on the input, deliver the insulin into the patient by the pump mechanism.

### BRIEF DESCRIPTION OF THE DRAWINGS

In the drawings, like reference characters generally refer to the same parts throughout the different views. In the following description, various embodiments of the present disclosure are described with reference to the following drawings, in which:

FIG. 1 illustrates an example of a system according to embodiments of the present disclosure;

FIG. 2 illustrates an example of a drug delivery system according to embodiments of the present disclosure; and

FIG. 3 illustrates a process flow according to embodiments of the present disclosure.

The drawings are not necessarily to scale. The drawings are merely representations, not intended to portray specific parameters of the disclosure. The drawings are intended to depict exemplary embodiments of the disclosure, and therefore are not be considered as limiting in scope. Furthermore, certain elements in some of the figures may be omitted, or illustrated not-to-scale, for illustrative clarity. Still furthermore, for clarity, some reference numbers may be omitted in certain drawings.

### DETAILED DESCRIPTION

Systems, devices, and methods in accordance with the present disclosure will now be described more fully hereinafter with reference to the accompanying drawings, where one or more embodiments are shown. The systems, devices, and methods may be embodied in many different forms and are not to be construed as being limited to the embodiments set forth herein. Instead, these embodiments are provided so the disclosure will be thorough and complete, and will fully convey the scope of methods and devices to those skilled in the art. Each of the systems, devices, and methods disclosed herein provides one or more advantages over conventional systems, components, and methods.

As noted above, conventional drug delivery devices require a high level of user interaction and/or communication with external interface devices, sometimes termed personal diabetes managers (PDM). For example, with continuous subcutaneous insulin infusion (CSII) pump devices, a user needs to set his/her time-dependent basal profiles, correction factors, insulin to carbohydrate issues, and a multitude of other clinical parameters before he/she can begin utilizing the system. The majority of commonly used CSII pump devices are factory configured and cannot be modified except by the PDM. This may act as a barrier for some patients.

Embodiments of the present disclosure provide a disposable drug delivery device that can provide insulin therapy to users without need for additional user actions beyond filling of insulin in the pump and placing the system onto the user's body. Specifically, the drug delivery device is able to execute all functions required for reliable insulin delivery without an external PDM. As will be described in greater detail herein, some non-limiting functions performed by the drug delivery device, without PDM intervention, may include defining insulin therapy settings, waking the drug delivery device from a dormant state to an active state, and controlling needle insertion, either with manual or passive activation.

Embodiments of the present disclosure may also provide approaches for modifying insulin delivery rates through simple physical interactions with the drug delivery device after needle insertion, without a PDM. In one non-limiting example, the drug delivery device may include a hand-operated tool or mechanism on the drug delivery device for operation and control. This may add flexibility to fine-tune basal rates between minimum and maximum limits, for example. Adjustments may also be achieved by providing a set of simple, reusable peripherals which, when held close to the pump, can alter the delivery rate.

As used herein, the algorithms or computer applications that manage blood glucose levels and insulin therapy may be referred to as an “artificial pancreas” algorithm-based system, or more generally, an artificial pancreas (AP) application. An AP application may be programming code stored in a memory device and that is executable by a processor, controller or computer device. Examples of an AP application as discussed herein provide automatic control/operation of drug delivery devices without the use of an external interface, such as a PDM. Embodiments of the present disclosure may also provide approaches for modifying behaviors of an AP application through physical or other interactions.

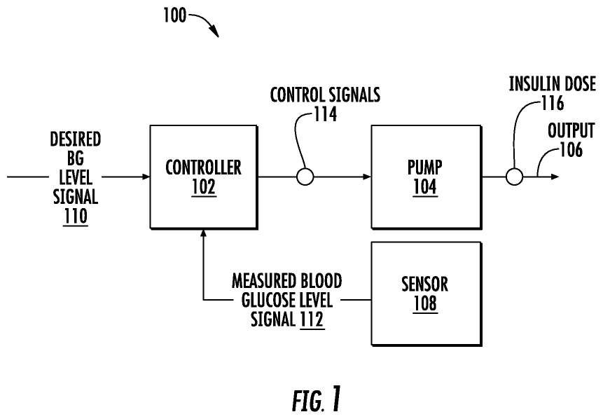

FIG. 1 illustrates a simplified block diagram of an example of an AP system (hereinafter “system”) 100. The example system 100 may include a controller 102, a pump mechanism 104 (hereinafter “pump 104”), and a sensor 108. The controller 102, pump 104, and sensor 108 may be communicatively coupled to one another via a wired or wireless communication path. For example, each of the controller 102, the pump 104 and the sensor 108 may be equipped with a wireless radio frequency transceiver operable to communicate via one or more communication protocols, such as Bluetooth®, or the like. The sensor 108 may be a glucose monitor such as, for example, a continuous glucose monitor. The sensor 108 may, for example, be operable to measure blood glucose (BG) values of a user to generate the measured BG level signal 112.

As shown in the example, the controller 102 may receive a desired BG level signal 110, which may be a first signal, indicating a desired BG level or range for a user. The desired BG level signal 110 may be received from a user interface to the controller or other device, such as a mechanical triggering device (e.g., a dial), or by an algorithm that automatically determines a BG level for a user. The sensor 108 may be coupled to the user and operable to measure an approximate value of a BG level of the user. The measured BG value, the measured BG level, the measured BG level value, or the approximate measured value of the actual BG level are only approximate values of a user's BG level and it should be understood that there may be errors in the measured BG levels or values. The terms measured BG value and approximate measured value of the BG level may be used interchangeably throughout the specification and drawings. In response to the measured BG level or value, the sensor 108 may generate a signal indicating the measured BG value. As shown in the example, the controller 102 may also receive from the sensor 108 via a communication path, a measured BG level signal 112, which may be a second signal, indicating an approximate measured value of the measured BG level of the user.

Based on the desired BG level signal 110 and the measured BG level signal 112, the controller 102 may generate one or more control signals 114 for directing operation of the pump 104. For example, one of the control signals 114 may cause the pump 104 to deliver a specified amount of insulin 116 to a user via output 106. The specified amount of insulin 116 may, for example, be determined based on a difference between the desired BG level signal 110 and the actual BG signal level 112. The specified amount of insulin may be determined as an appropriate amount of insulin to drive the measured BG level of the user to the desired BG level. Based on operation of the pump 104, as determined by the control signals 114, the user may receive the insulin 116 from the pump 104. The system 100 may operate as a closed-loop system or may operate as an open-loop system.

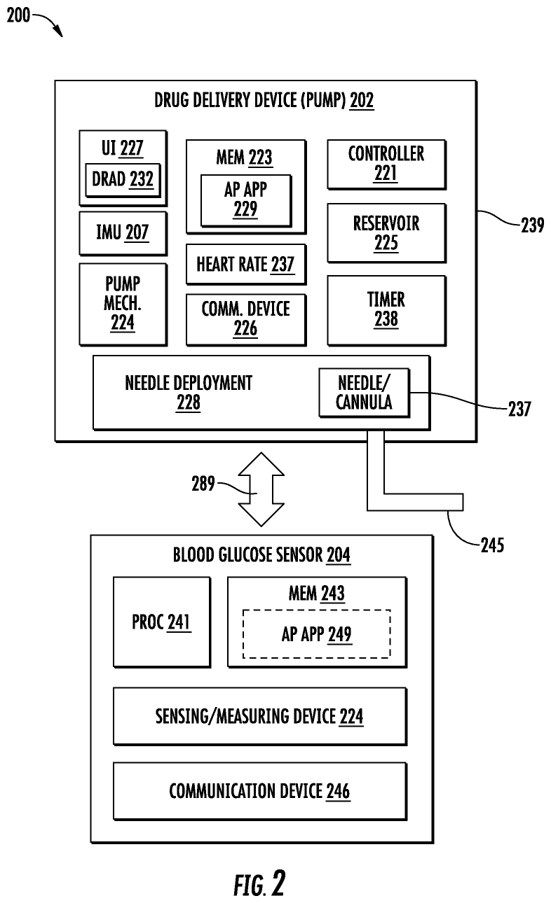

FIG. 2 illustrates an example of a drug delivery system 200. Various examples of the drug delivery system 200 include a wearable drug delivery device that may operate to manage treatment of a diabetic user according to a diabetes treatment plan. The diabetes treatment plan may include a number of parameters related to the delivery of insulin that may be determined and modified without the use of an external management device.

As shown, the drug delivery system 200 may include a drug delivery device 202 and a blood glucose sensor 204. In this embodiment, the drug delivery device 202 may be a wearable or on-body drug delivery device attached to the skin of the user. As shown, the drug delivery device 202 may include an inertial measurement unit (IMU) 207, a pump mechanism 224 that may, in some examples, be referred to as a drug extraction mechanism or component, and a needle deployment component 228. In various examples, the pump mechanism 224 may include a pump or a plunger (not shown). The needle deployment component 228 may include a needle/cannula 237, and any other fluid path components for coupling the stored liquid drug in the reservoir 225 to the user. The cannula 237 may form a portion of the fluid path component coupling the user to the reservoir 225. After the needle deployment component 228 has been activated, a fluid path (not shown) to the user is provided, and the pump mechanism 224 may expel the liquid drug from the reservoir 225 to deliver the liquid drug to the user via the fluid path. The fluid path may, for example, include tubing (not shown) coupling the drug delivery device 202 to the user (e.g., tubing coupling the cannula 237 to the reservoir 225). The drug delivery device 202 may further include a controller 221 and a communications interface device 226. The controller 221 may be implemented in hardware, software, or any combination thereof. The controller 221 may, for example, be a processor, a logic circuit or a microcontroller coupled to a memory. The controller 221 may maintain a date and time as well as other functions (e.g., calculations or the like) performed by processors. The controller 221 may be operable to execute an artificial pancreas algorithm stored in the memory that enables the controller 221 to direct operation of the drug delivery device 202. For example, the controller 221 may receive an input from a mechanical triggering device 245 operable to change between a first configuration and a second configuration to control a basal insulin delivery rate and/or deployment of the needle 237 to deliver the insulin into the patient. In addition, the controller 221 may be operable to receive data or information indicative of the activity of the user from the IMU 207, as well as from any other sensors, such as the blood glucose sensor 204.

In another example, the controller 221 may be communicatively coupled to the pump mechanism 224 and to the mechanical triggering device 245. The controller 221 is operable to receive an input from the mechanical triggering device 245, wherein the input indicates an automated insulin delivery (AID) application setting. Based on the AID application setting, the controller 221 may modify the behavior of the AID delivery application and resulting amount of the liquid drug to be delivered by the pump mechanism 224.

The controller 221 may process the data from the IMU 207 or any other coupled sensor to determine if an alert or other communication is to be issued to the user and/or a caregiver of the user, or if an operational mode of the drug delivery device 202 is to be adjusted. The controller 221 may provide the alert, for example, through the communications interface device 226. The communication link provided by the communications interface device 226 may include any wired or wireless communication link operating according to any known communications protocol or standard, such as Bluetooth, NFC, or a cellular standard.

In some embodiments, the blood glucose sensor 204 may be, for example, a continuous glucose monitor (CGM). The blood glucose sensor 204 may be physically separate from the drug delivery device 202 or may be an integrated component within a same housing 239 thereof. The blood glucose sensor 204 may provide the controller 221 with data indicative of measured or detected blood glucose levels of the user.

The drug delivery system 200 may be operable to implement an AP application 229 that includes functionality to provide insulin therapy to users without the need for additional user actions beyond filling of insulin in the pump and placing the system onto the user's body. The AP application 229 may further include functionality to define insulin therapy settings, wake the drug delivery device 202 from a dormant state to an active state, and control needle insertion, either with manual or passive activation. The AP application 229 may further include functionality to modify insulin delivery rates through simple physical interactions with the drug delivery device, after needle insertion, and without a PDM.

The drug delivery device 202 may frequently be referred to as a pump, or an insulin pump, in reference to the operation of expelling a drug from the reservoir 225 for delivery of the drug to the user. In an example, the drug delivery device 202 may include a reservoir 225 for storing the liquid drug, such as insulin, the needle/cannula 237 for delivering the drug into the body of the user (which may be done subcutaneously, intraperitoneally, or intravenously), and the pump mechanism 224, or other drive mechanism, for transferring the drug from the reservoir 225, through the needle/cannula 237, and into the user. The reservoir 225 may be configured to store or hold a liquid or fluid, such as insulin, morphine, or another therapeutic drug. The pump mechanism 224 may be fluidly coupled to the reservoir 225, and communicatively coupled to the controller 221. The drug delivery device 202 may also include a power source (not shown), such as a battery, a piezoelectric device, or the like, for supplying electrical power to the pump mechanism 224 and/or other components (such as the controller 221, memory 223, and the interface communication device 226) of the drug delivery device 202. Although also not shown, an electrical power supply for supplying electrical power may similarly be included in the blood glucose sensor 204.

In an example, the blood glucose sensor 204 may be a device communicatively coupled to the controller 221 and may be operable to measure a blood glucose value at a predetermined time interval, such as approximately every 5 minutes, 10 minutes, or the like. The blood glucose sensor 204 may provide a number of blood glucose measurement values to the AP application 229.

In some embodiments, the IMU 207 of the drug delivery device 202 may be operable to detect various motion parameters (e.g., acceleration, deceleration, speed, orientation, such as roll, pitch, yaw, compass direction, or the like) that may be indicative of the activity of the user. For example, the IMU 207 may output signals in response to detecting motion of the drug delivery device 202 that is indicative of a status of any physical condition of the user, such as, for example, a motion or position of the user. Based on the detected activity of the user, the drug delivery device 202 may adjust operation related to drug delivery, for example.

In some embodiments, the drug delivery device 202 may, when operating in a normal mode of operation, provide insulin stored in the reservoir 225 to the user based on information (e.g., blood glucose measurement values, target blood glucose values, insulin on board, prior insulin deliveries, time of day, day of the week, inputs from an inertial measurement unit, global positioning system-enabled devices, Wi-Fi-enabled devices, or the like) provided by the blood glucose sensor 204 or other functional elements on drug delivery device 202. For example, the drug delivery device 202 may contain analog and/or digital circuitry that may be implemented as the controller 221 for controlling the delivery of the drug or therapeutic agent. The circuitry used to implement the controller 221 may include discrete, specialized logic and/or components, an application-specific integrated circuit, a microcontroller or processor that executes software instructions, firmware, programming instructions or programming code enabling, for example, an AP App 229 stored in memory 223, or any combination thereof. For example, the controller 221 may execute a control algorithm and other programming code that may make the controller 221 operable to cause the pump to deliver doses of the drug or therapeutic agent to a user at predetermined intervals or as needed to bring blood glucose measurement values to a target blood glucose value. The size and/or timing of the doses may be pre-programmed, for example, into the AP application 229 by the user or by a third party (such as a health care provider, a parent or guardian, a manufacturer of the wearable drug delivery device, or the like) using a wired or wireless link.

In some embodiments, the blood glucose sensor 204 may include a processor 241, a memory 243, a sensing or measuring device 244, and a communication device 246. The memory 243 may store an instance of an AP application 249 as well as other programming code and be operable to store data related to the AP application 249.

In various embodiments, the sensing/measuring device 244 of the blood glucose sensor 204 may include one or more sensing elements, such as a blood glucose measurement element, a heart rate monitor, a blood oxygen sensor element, or the like. The processor 241 may include discrete, specialized logic and/or components, an application-specific integrated circuit, a microcontroller or processor that executes software instructions, firmware, programming instructions stored in memory (such as memory 243), or any combination thereof. For example, the memory 243 may store an instance of an AP application 249 that is executable by the processor 241.

Instructions for determining the delivery of the drug or therapeutic agent (e.g., as an adjustable basal or bolus dosage) to the user (e.g., the size and/or timing of any doses of the drug or therapeutic agent) may originate locally by the drug delivery device 202 or may originate remotely and be provided to the drug delivery device 202. In an example of a local determination of drug or therapeutic agent delivery, programming instructions, such as an instance of the AP application 229, stored in the memory 223 that is coupled to the drug delivery device 202 may be used to make determinations by the drug delivery device 202. In addition, the drug delivery device 202 and the blood glucose sensor 204 may communicate via one or more communication links 289.

The drug delivery device 202 may also include a user interface 227. As will be described in greater detail herein, the user interface 227 may include any a delivery rate adjustment device (DRAD) 232 for the user to input data to the drug delivery device 202, such as, for example, a dial button, a knob, a dial, a switch, a touch-screen display, or any other user interaction component. The user interface 227 may include any mechanism for the drug delivery device 202 to relay data to the user and may include, for example, a numbered dial or knob, a display, a touch-screen display, or any means for providing a visual, audible, or tactile (e.g., vibrational) output (e.g., as an alert). In some embodiments, the DRAD 232 may allow the user to both give and receive data to the drug delivery device 202. The user interface 227 may also include a number of additional components not specifically shown in FIG. 2 for the sake brevity and explanation. For example, the user interface 227 may include one or more user input or output components for receiving inputs from or providing outputs to a user or a HCP, a display that outputs a visible alert, a speaker that outputs an audible alert, or a vibration device that outputs tactile indicators to alert a user or a caregiver of a potential activity or operational mode, a power supply (e.g., a battery), and the like. Inputs to the user interface 227 may, for example, be a via a fingerprint sensor, a tactile input sensor, a button, a touch screen display, a switch, or the like. In general, a user may generate instructions that may be stored as user preferences in a memory, such as memory 223, that specify when the system 200 is to enter the activity mode of operation.

In some embodiments, the system 200 is operable to set or define insulin therapy settings, for example, without the use of a PDM. The system 200 can be designed to allow users or HCPs to choose the desired basal rates within particular constraints and limits of insulin dosing without a PDM. In one passive insulin therapy setting approach, the system 200 can provide pre-configured drug delivery devices 202 that can be selected by the HCP or user to deliver a fixed insulin therapy. For example, different product packaging may indicate that the drug delivery device 202 will deliver 0.6 U/h over its life, versus a drug delivery device 202 that will deliver 0.3 U/h over its life. In one active insulin therapy setting approach, users or HCPs can receive a set of non-configured drug delivery devices 202 that can then be individually configured once the drug delivery devices 202 are received by the user or HCP. In various examples, the drug delivery devices 202 can be set individually for each infusion set or can be set per group, such as per each individual box containing a plurality of drug delivery devices 202.

In another embodiment, the HCP can be the primary originator of the individually configured drug delivery device 202, and be provided with a mechanism that provides HCP-only access, such as a validated account, or registered device, to set insulin therapy configurations per system. This will reduce the possibility of user error due to laymen interactions. Alternatively, a pharmacist or other medically controlled distribution channel could pre-configure the drug delivery device 202.

In another embodiment, insulin therapy settings may be defined as an interaction with manufacturers. For example, the users or HCPs can order a set of drug delivery devices 202 that are pre-configured to a set therapy rate at time of order, and receive individualized systems shipped directly from the manufacturer. Although non-limiting, the users themselves can request modified drug delivery devices 202 through an application or a web portal, or the HCPs can order individualized drug delivery devices 202 for each user.

In some embodiments, the system 200 is operable to trigger the drug delivery device 202 from a dormant state to an active state, for example, without the use of a PDM. In one passive activation example of the drug delivery device 202, mechanisms or tools may be added to the system 200, the drug delivery device 202, and/or packaging for the drug delivery device 202 to initiate activation of the system 200. In one embodiment, biasing or pulling the mechanical triggering device 245 causes the drug delivery device 202 to “wake-up.” Although non-limiting, another mechanism or a component on the drug delivery device 202 may be used to determine that the drug delivery device 202 has exited a sterile or controlled environment, such as packaging of the drug delivery device 202. In another example, a mechanism or component on the drug delivery device 202, such as a sensor, can also detect placement of the drug delivery device 202 on a body. In yet another example, the packaging may have specific connections with the device, which may be disposable elements, such as a needle cap or primary system, which when broken or altered, may signal that the drug delivery device 202 has exited the packaging and is therefore ready to be activated.

One user-guided activation example may include a simple, reusable peripheral device utilized by the user or HCP to wirelessly indicate device activation readiness. This peripheral device may be provided by the manufacturer, and emit a specific signal, which activates the drug delivery device 202 once the drug delivery device 202 receives the signal for a certain duration of time.

In some embodiments, the system 200 may further control operation of the needle/cannula 237 of the drug delivery device 202 with only manual or passive activation. In this embodiment, the system 200 can be designed to both activate the drug delivery device 202 and cause insertion of the needle/cannula, without PDM intervention. In one passive needle insertion approach, a mechanism or component on the drug device 202, such as the mechanical triggering device 245, can activate a timer 238 to automatically insert the needle/cannula 237 once a timing cycle expires. For example, a countdown audible beep or tone, or a variation in the frequency of the audible beep or tone, may indicate to the user when the needle 237 will be inserted. An LED or light on the drug delivery device 202 may be further provided to indicate to the user that the needle 237 will be inserted once the drug delivery device 202 activated, using a similar countdown or variation in light emission frequency or wavelength.

In another example, needle insertion can be triggered when the system 200 determines that the drug delivery device 202 has been placed on the body. For example, a heart rate monitor, a blood oxygen sensor element, a light sensor, or the like, may indicate placement on the skin of the user. Once a positive (or negative) detection is achieved, the timer 238, which may include auditory, or visual aids, etc., may be activated.

In one active needle insertion approach, a mechanism on the drug delivery device 202 can be engaged by the user to indicate to the controller 221 that the needle 237 is ready to be inserted. For example, the user may interact with a specific element of each device, such as the mechanical triggering device 245, which may comprise a ripcord or the removable tab, to initiate needle insertion.

In another embodiment, the mechanical triggering device 245 may be directly physically coupled to a trigger block and/or a trigger lever (not shown) of the needle deployment component 228. The trigger block and the trigger lever may hold the needle/cannula 237 in place, preventing it from being deployed. Once the mechanical triggering device 245 is pulled or removed, the trigger lever will be moved, enabling the trigger block and the needle/cannula 237 to be biased towards the skin of the patient in response to a force from one or more springs, for example.

In another example, a reusable simple peripheral device can be provided by the manufacturer, and a user with the drug delivery device 202 already on his/her body can bring the peripheral device close to the pump, which can either initiate the insertion or activate a timer for insertion. In yet another example, a low-cost “wand” may be employed to trigger needle insertion, and may comprise, for example, a magnet or piezoelectric element.

In some embodiments, the system 200 may further allow modification of basal insulin delivery rates or bolus delivery amounts through simple physical interactions with the drug delivery device 202, after needle insertion, and without a PDM. In one non-limiting example, a fill needle (tip) of the drug delivery device 202 may include a keying feature, which interfaces to a lock receptacle of the drug delivery device 202. The patient may dial therapy based on gradations on the drug delivery device 202 or syringe. Alternatively, the DRAD 232 of the user interface 227 may comprise a knob or dial with numbered gradations indicating a particular basal delivery rate, and/or a second knob or dial with numbered gradations indicating a particular bolus delivery mount, either of which may be tuned by the user.

In another non-limiting example, the DRAD 232 may be a needle cap of the drug delivery device 202, wherein the needle cap may be used as a key to set a basal rate. During use, the needle cap may be brought into place, and then turned/rotated to set the basal rate. Alternatively, with the needle cap removed, a feature on the needle cap may interface with a lock/receptacle on the drug delivery device 202 to allow a user to dial in a basal rate using numbered indicators.

In another non-limiting example, when a detachable needle insertion mechanism is utilized, the needle insertion mechanism may include a dial or knob to set a basal rate. As mentioned above, the DRAD 232 of the user interface 227 may include the dial or knob. Alternatively, the needle insertion mechanism may be specific to one set or predetermined basal rate (e.g., 3 U/hr basal needle insertion mechanism, different key features, and other visual cues to a user indicating which basal rate their device is configured have). In another non-limiting embodiment, the mechanical triggering device 245 of the drug delivery device 202 is a ripcord that can be adjusted and pulled/removed from the drug delivery device 202 to set a basal rate.

In another non-limiting embodiment, the user may obtain a pre-filled syringe or a standard syringe, which may be filled a set amount to deliver insulin over the duration of the life of the device, such as over 3 days. In one example, if the user uses 30 U of insulin a day, the drug delivery device would automatically set a basal rate of 1.25 U/hr for three days when 90 U+/−X U are filled into the drug delivery device. This serves to minimize insulin waste and remove steps for the user to set basal rate.

In another non-limiting embodiment, the drug delivery device 202 may include an embedded subscriber identity module (eSIM), which connects to a patient network to identify the patient and his/her therapy needs. The electronics of the drug delivery device 202 may make adjustments to delivery rate and could also be verified within the same network.

In another non-limiting embodiment, the AP application 229 may set a configured therapy for the drug delivery device 202. BLE, RFID, QR Code ID, etc., may adjust therapy of the drug delivery device 202. In some embodiments, an HCP may have a portal to monitor patients, and can therefore see when a patient activates a device. The HCP may then set the basal rate for a given lot/box of devices, or may do it in real-time during device wake up or activation.

In another non-limiting embodiment, the drug delivery device 202 may be configured by the manufacturer. For example, drug delivery device 202 may be programmed at the manufacturer into groupings, or programmed to have settings specific to particular users. This may be advantageous with a model in which the drug delivery device 202 is built on demand, e.g., based on re-order.

In another non-limiting embodiment, the drug delivery device 202 may be configured by a pharmacy based on a particular prescription for the patient. This can be done by programming the drug delivery device 202 through outer packaging (e.g., direct to device via NFC, RFID, BLE, vibration, audible frequency>20 kHz, etc.). Pharmacy configuration may also be accomplished by programming a smart box/insulin vial, which conveys the basal rate to the drug delivery device 202 as it is awakened, or by creating a label with QR code that the user's smart device uses to code the drug delivery device 202.

In another non-limiting embodiment, basal settings can be transmitted between multiple drug delivery devices 202, again, without the use of a PDM. This may be performed for a user's soon-to-expire or expired device so the last basal setting can be transmitted to the new device via BLE, NFC, etc.

In another non-limiting embodiment, a biometric ID scanner may be used to set a patient's unique basal rate based on patient data stored in a database accessible via the cloud or a patient portal. For example, a scanner to identify a finger print on the drug delivery device 202, a voice recognition element, photoplethysmogram (PPG) to look at physiological characteristics, a blood type sensor, or other sensor to identify unique patient characteristics, which characteristics may be tied to a unique patient serial number, which may, in turn, be used to identify the patient's particular basal insulin needs stored in the database and accessible via the cloud or a patient portal.

In another non-limiting embodiment, the drug delivery device 202 may be shipped in a smart box, which may include any variety of input communication components, which may be configured within bulk packaging or blister pack to set basal rate. Although non-limiting, the input communication component may be a dial, pull tab, NFC transmitter, a wand, a key fob, a flex circuit (e.g., button), etc., which may be set at the time of activation. For example, the basal rate may be selected by adjustment of a dial of the smart box, which may then be communicated to the drug delivery device when the drug delivery device transitions from an inactive state to an active state.

In another non-limiting embodiment, the drug delivery device 202 may receive an indication of basal rate through a series of flashing lights. For example, pulses of short and long flashes (e.g., Morse Code) may be generated by a dongle or smart device (e.g., phone or watch) and received at a light detector/sensor of the drug delivery device 202. The controller 221 is operable to process the sensor output to control basal rates.

Any of the drug delivery systems, devices, and/or pumps disclosed herein, can be an OmniPod® (Insulet Corporation, Acton, Mass.) insulin delivery device and/or can be any of the drug delivery systems, devices, and/or pumps described in U.S. Pat. Application Ser. No. 63/150,871, which is incorporated herein by reference in its entirety and for all purposes.

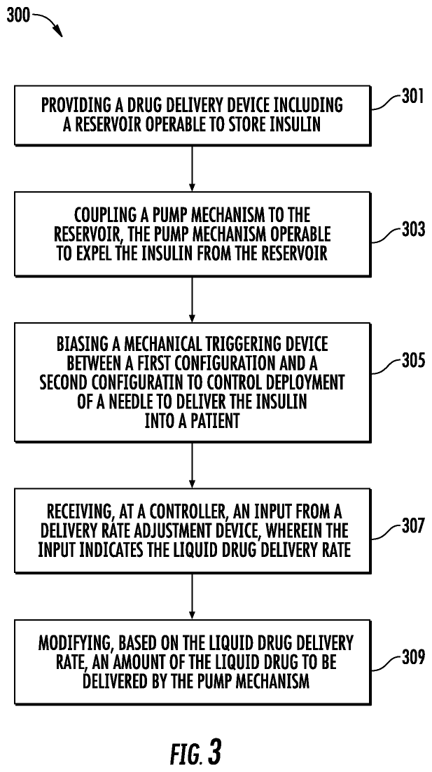

FIG. 3 illustrates an example process 300 implemented by the system 200 according to the present disclosure. At block 301, the process 300 may include providing a drug delivery device including a reservoir operable to store insulin. In some embodiments, the drug delivery device is a wearable drug delivery system that is attachable to the skin of a user.

At block 303, the process 300 may include coupling a pump mechanism to the reservoir, the pump mechanism being operable to expel the insulin from the reservoir. In some embodiments, the drug delivery device may further include a needle deployment component, which may include a needle and cannula, and any other fluid path components for coupling the stored liquid drug in the reservoir to the user.

At block 305, the process 300 may include biasing a mechanical triggering device between a first configuration and a second configuration to control deployment of a needle to deliver the insulin into a patient. It will be appreciated that the deployment of the needle may be controlled or initiated without communication between the drug delivery device and an external interface device, such as a PDM.

In some embodiments, the mechanical triggering device extends outside a housing containing the reservoir and the pump mechanism, wherein the mechanical triggering device is a ripcord or a removable tab. In some embodiments, the process 300 may further include automatically deploying the needle at an expiration of a timing cycle. In some embodiments, the timing cycle is initiated in response to engagement of the ripcord or the removable tab. In some embodiments, the needle deployment component is released in response to engagement of the ripcord or the removable tab.

At block 307, the process 300 may optionally include receiving, at a controller, an input from a delivery rate adjustment device, wherein the input indicates the liquid drug delivery rate. In some embodiments, the delivery rate adjustment device may be a dial button, a knob, a dial, a switch, a touch-screen display, or any other user interaction component. In some embodiments, the delivery rate adjustment device may be a component of a user interface. In various embodiments, the input from the delivery rate adjustment device may be received before or after deployment of the deployment of the needle.

At block 309, the process 300 may optionally include modifying, based on the liquid drug delivery rate, an amount of the liquid drug to be delivered by the pump mechanism.

In some embodiments, the process 300 may include communicatively coupling the controller to the pump mechanism and to the mechanical triggering device or delivery rate adjustment device of the user interface.

In some embodiments, the process 300 described above may modify parameters in an AP application in place of clinical glucose control parameters. For instance, physical interactions with the drug delivery device may determine that the AP application may operate in a manner that would deliver an increased amount of insulin, or enter a mode where it will only reduce possible insulin deliveries but not increase insulin delivery above the user's standard care. Physical interactions with the drug delivery device may also determine that the AP application may only be active at certain portions of the day, such as during daytime hours, or following certain periods of use, such as starting automated delivery after at least 36 hours of use. In one non-limiting example, the user may not immediately enter automated mode, but instead use the system under manual mode for a period of time (e.g., the first 24, 36, 48 hours etc.). The user may use the DRAD on the drug delivery device to manually control basal delivery rates, for example. This may be useful when the user does not have confidence that the AID system is working properly, or when the user wishes to manually tune operation of the system over the initial period of time.

In another exemplary embodiment, the process 300 described above may also provide a passive “advertisement” of one or more statuses of the drug delivery device, such as remaining liquid drug capacity, activation/deactivation events, drug delivery rates, measured BG values, error states, how long the drug delivery device is being used, how many user interactions were executed, and others. The status information may be communicated via standard communication methods such as by a Bluetooth low-energy or RF signal. In one non-limiting example, the passive advertisement can be encoded by a specific application programming interface (API), such as communications interface device 226, wherein any external device that has access to this API will be able to receive the status information of the drug delivery device. In this example, a HCP may gain visibility to patterns in patient use of the drug delivery device, thus improving the capability for ongoing patient care. Moreover, the API may allow a 3^{rd} party device to receive and utilize data from the drug delivery device to provide, for example, an optional visual display of the various device statuses.

The techniques described herein for a drug delivery system (e.g., the systems 100, 200 or any components thereof) may be implemented in hardware, software, or any combination thereof. Any component as described herein may be implemented in hardware, software, or any combination thereof. For example, the systems 100 and 200 or any components thereof may be implemented in hardware, software, or any combination thereof. Software related implementations of the techniques described herein may include, but are not limited to, firmware, application specific software, or any other type of computer readable instructions that may be executed by one or more processors. Hardware related implementations of the techniques described herein may include, but are not limited to, integrated circuits (ICs), application specific ICs (ASICs), field programmable arrays (FPGAs), and/or programmable logic devices (PLDs). In some examples, the techniques described herein, and/or any system or constituent component described herein may be implemented with a processor executing computer readable instructions stored on one or more memory components.

Some examples of the disclosed devices may be implemented, for example, using a storage medium, a computer-readable medium, or an article of manufacture which may store an instruction or a set of instructions that, if executed by a machine (i.e., processor or controller), may cause the machine to perform a method and/or operation in accordance with examples of the disclosure. Such a machine may include, for example, any suitable processing platform, computing platform, computing device, processing device, computing system, processing system, computer, processor, or the like, and may be implemented using any suitable combination of hardware and/or software. The computer-readable medium or article may include, for example, any suitable type of memory unit, memory, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, programming code, and the like, implemented using any suitable high-level, low-level, object-oriented, visual, compiled and/or interpreted programming language. The non-transitory computer readable medium embodied programming code may cause a processor when executing the programming code to perform functions, such as those described herein.

Certain examples of the present disclosed subject matter were described above. It is, however, expressly noted that the present disclosed subject matter is not limited to those examples, but rather the intention is that additions and modifications to what was expressly described herein are also included within the scope of the disclosed subject matter. Moreover, it is to be understood that the features of the various examples described herein were not mutually exclusive and may exist in various combinations and permutations, even if such combinations or permutations were not made express herein, without departing from the spirit and scope of the disclosed subject matter. In fact, variations, modifications, and other implementations of what was described herein will occur to those of ordinary skill in the art without departing from the spirit and the scope of the disclosed subject matter. As such, the disclosed subject matter is not to be defined only by the preceding illustrative description.

Program aspects of the technology may be thought of as “products” or “articles of manufacture” typically in the form of executable code and/or associated data that is carried on or embodied in a type of machine readable medium. Storage type media include any or all of the tangible memory of the computers, processors or the like, or associated modules thereof, such as various semiconductor memories, tape drives, disk drives and the like, which may provide non-transitory storage at any time for the software programming. It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of example examples has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

What is claimed is:

1. A wearable drug delivery device, comprising:
a reservoir configured to store a liquid drug;
a pump mechanism coupled to the reservoir and operable to expel the liquid drug from the reservoir; and
a mechanical triggering device engageable by a user, the mechanical triggering device operable to change between a first configuration and a second configuration to control deployment of a needle to deliver the liquid drug into a patient.

2. The wearable drug delivery device of claim 1, further comprising a controller communicatively coupled to the pump mechanism and to a delivery rate adjustment device, wherein the controller is operable to:
receive an input from the delivery rate adjustment device, wherein the input indicates a liquid drug delivery rate; and
based on the liquid drug delivery rate, modify an amount of the liquid drug to be delivered by the pump mechanism.

3. The wearable drug delivery device of claim 2, wherein the controller is further operable to:
receive an input indicating an automated insulin delivery (AID) application setting; and
based on the AID application setting, modify a behavior of the AID application and resulting amount of the liquid drug to be delivered by the pump mechanism.

4. The wearable drug delivery device of claim 2, further comprising a housing containing the reservoir and the pump mechanism, wherein the mechanical triggering device is a ripcord or a removable tab extending outside the housing.

5. The wearable drug delivery device of claim 4, further comprising a timer, wherein at an expiration of a timing cycle, the needle is automatically deployed, wherein engagement of the ripcord or the removable tab causes the timer to begin the timing cycle.

6. The wearable drug delivery device of claim 4, further comprising a needle deployment component, wherein engagement of the ripcord or the removable tab causes the needle deployment component to insert the needle into the patient.

7. The wearable drug delivery device of claim 2, further comprising a user interface, wherein the delivery rate adjustment device is a dial or a knob of the user interface.

8. A method, comprising:
providing a drug delivery device including a reservoir operable to store a liquid drug;
coupling a pump mechanism to the reservoir, the pump mechanism operable to expel the liquid drug from the reservoir; and
biasing a mechanical triggering device between a first configuration and a second configuration to control deployment of a needle to deliver the liquid drug into a patient.

9. The method of claim 8, further comprising:
communicatively coupling a controller to the pump mechanism and to a delivery rate adjustment device;
receiving, at the controller, an input from the delivery rate adjustment device, wherein the input indicates a liquid drug delivery rate; and
modifying, based on the liquid drug delivery rate, an amount of the liquid drug to be delivered by the pump mechanism.

10. The method according to claim 9, further comprising adjusting, after deployment of the needle, the liquid drug delivery rate by adjusting the delivery rate adjustment device, wherein the delivery rate adjustment device is an adjustable dial of a user interface of the drug delivery device.

11. The method according to claim 8, further comprising extending the mechanical triggering device outside a housing containing the reservoir and the pump mechanism, wherein the mechanical triggering device is a ripcord or a removable tab.

12. The method according to claim 11, further comprising automatically deploying the needle at an expiration of a timing cycle.

13. The method according to claim 12, further comprising initiating the timing cycle in response to engagement of the ripcord or the removable tab.

14. The method according to claim 12, further comprising releasing, in response to engagement of the ripcord or the removable tab, a needle deployment component to insert the needle into the patient.

15. The method according to claim 8, further comprising controlling the deployment of the needle without communication between the drug delivery device and an external interface device.

16. A non-transitory computer readable medium embodied with programming code executable by a processor, and the processor when executing the programming code is operable to perform functions, including functions to:
receive an input from a mechanical triggering device communicably connected with a reservoir and a pump mechanism of a drug delivery device, wherein the input indicates deployment of a needle to deliver a liquid drug from the reservoir to a patient; and
based on the input, deliver the liquid drug into the patient by the pump mechanism.

17. The non-transitory computer readable medium of claim 16, further embodied with programming code executable by the processor, and the processor when executing the programming code is operable to performing functions to automatically deploy the needle at an expiration of a timing cycle.

18. The non-transitory computer readable medium of claim 17, further embodied with programming code executable by the processor, and the processor when executing the programming code is operable to initiate the timing cycle in response to engagement of the mechanical triggering device.

19. The non-transitory computer readable medium of claim 16, further embodied with programming code executable by the processor, and the processor when executing the programming code is operable to release, in response to engagement of the mechanical triggering device, a needle deployment component to insert the needle into the patient.

20. The non-transitory computer readable medium of claim 16, further embodied with programming code executable by the processor, and the processor when executing the programming code is operable to:
receive an input from a delivery rate adjustment device of a user interface of the drug delivery device, wherein the input indicates a liquid drug delivery rate; and
modify, based on the liquid drug delivery rate, an amount of the liquid drug to be delivered by the pump mechanism.

21. The non-transitory computer readable medium of claim 20, further embodied with programming code executable by the processor, and the processor when executing the programming code is operable to control the liquid drug delivery rate and to control the deployment of the needle without communication between the drug delivery device and an external interface device.

22. The non-transitory computer readable medium of claim 16, further embodied with programming code executable by the processor, and the processor when executing the programming code is able to communicate a status of the pump mechanism to an external device, wherein the external device has access to a communication interface of the drug delivery device.
